(19) United States
(12) Patent Application Publication
(10) Pub. No.: US 2024/0393229 A1
(43) Pub. Date: Nov. 28, 2024

(54) METHOD FOR DETECTING AND QUANTIFYING FUNGI AND BACTERIA USING FLOW CYTOMETRY

(71) Applicant: MD INVENTION AGRO, Montredon-des-Corbières (FR)

(72) Inventor: Matthieu DUBERNET, NARBONNE (FR)

(73) Assignee: MD INVENTION AGRO, Montredon-des-Corbières (FR)

(21) Appl. No.: 18/694,984

(22) PCT Filed: Sep. 27, 2022
(86) PCT No.: PCT/IB2022/059196

§ 371 Date: Mar. 24, 2024

(30) Foreign Application Priority Data
Sep. 28, 2021 (FR) FR2110237

(51) Int. Cl. G01N 15/14; C12Q 1/24; G01N 15/10; G01N 21/64

(52) U.S. Cl. CPC G01N 15/1459; C12Q 1/24; G01N 21/6486; G01N 2015/1006; G01N 2015/1486

(57) ABSTRACT

The present invention relates to a method for the simultaneous detection and quantification of fungi and bacteria present in a divided solid containing cellulosic organic matter. According to the invention, to said sample before introducing into said cytometer, at least a first fluorochrome capable of binding to DNA and emitting, following an excitation, in a wavelength equal to or greater than 599 and equal to or greater than 657 nm is added.

### TECHNICAL FIELD

The present invention relates to a flow cytometry method which makes it possible to simultaneously detect and quantify the fungi and bacteria present in a solid in the divided state containing organic matter.

### PRIOR ART

A growing substrate, whether it is natural such as top soil, or artificial such as potting soil is a complex medium in which numerous species of bacteria and fungi live. The same applies for composts, manures and other amendment substrates. These bacteria and fungi may be useful for plant growth or on the contrary be pathogenic. It is furthermore known that the activity of soil microorganisms (especially bacteria and fungi) also has an action, in the presence of oxygen, on the emission of CO₂ by respiration and on that of methane. In any case, it is seen that it is important to be able to rapidly quantify soil bacteria and fungi.

The publication entitled “A rapid flow cytometry method to assess bacterial abundance in agricultural soil” by M. Bressan and published in the journal Applied Soil Ecology in 2015 (88 (2015) pages 60-68) states that the number of bacterial cells contained in a soil may be determined by flow cytometry using the SYBR Green fluorochrome. This publication suggests that the SYTOX-green fluorochrome could also be used. The difficulty of the analysis lies in the processing of the soil sample because the bacteria are frequently strongly bound to the soil particles. This publication states that the soil sample is diluted in an 85% sodium chloride solution and filtered at 0.22 μm. The mixture is homogenized by means of a vortex for 5 minutes at full speed. After a centrifugation to remove the largest particles, the supernatant is analyzed by flow cytometry. This method is not applied to fungi.

The publication entitled “Optimization of a Method To Quantify Soil Bacterial Abundance by Flow Cytometry” by B. Khalili published on Jul. 16, 2021 in the journal American Society for Microbiology states that it is possible to quantify the bacteria population contained in a soil sample by flow cytometry. The bacteria are extracted with a supernatant added to the Nicodenz® medium (which is obtained from a benzoic acid derivative on which three hydrophilic aliphatic chains have been grafted). This publication applies to a soil sample in which E. coli cells have been added. Therefore, it does not consist of a real soil sample. Furthermore, the publication says nothing about fungi.

The publication entitled “Schrödinger's microbes: Tools for distinguishing the living from the dead in microbial ecosystems” by J. B. Emerson and published in 2017 in the journal Microbiome (DOI 10.1186/s40168-017-0285-3) reviews all the techniques that can be used to determine whether a microorganism is live or not. It is observed that some fluorochromes do not make it possible to distinguish live organisms from dead ones. This is, for example, the case of propidium iodide in the case of particular proteobacteria and mycobacteria. The publication states that reagents may also be used for yeasts. In the case of yeasts, a fluorescent marker is used which binds with chitin (which forms the fungal cell wall) coupled with a marker which only emits fluorescence when the cell membrane and the metabolic function are intact. This publication does not specifically concern the study of soils, which are complex media.

The publication entitled “Specific and Rapid Enumeration of Viable but Nonculturable and Viable-Culturable Gram-Negative Bacteria by Using Flow Cytometry” by M. M. Taimu Khan published in August 2010 in the journal Applied and Environmental Microbiology, (pages 5088-5096 Vol. 76, No. 15) states that it is possible to enumerate latent bacterial cells by flow cytometry. The bacteria studied are as follows: Escherichia coli O157:H7, Pseudomonas aeruginosa, Pseudomonas syringae, and Salmonella enterica serovar Typhimurium; the fluorescent markers used are as follows: SYTO 9®, SYTO 13®, SYTO 17®, SYTO 40® and propidium iodide. This publication is not concerned with soils and does not mention fungi.

The publication entitled “Monitoring physiological status of GFP-tagged Pseudomonas fluorescens SBW25 under different nutrient conditions and in soil by flow cytometry” published in the journal FEMS Microbiology Ecology in 2004 (volume 51, Page 123-132) states that it is possible to determine the quantity of Pseudomonas—bacteria useful for plant growth—by labelling the bacteria with a fluorescent gene.

The publication entitled “Flow cytometry as a tool to assess the effects of gamma radiation on the viability, growth and metabolic activity of fungal spores” by N. Mesquita and published in the journal International Biodeterioration & Biodegradation in 2013 (Volume 84, page 250-257) states that it possible to determine the viability of Penicillium chrysogenum, Aspergillus nidulans and Aspergillus niger spores by flow cytometry. The markers used are propidium iodide and dihydroethidium. Dihydroethidium is inserted in the DNA of these cells, staining the nucleus red. This publication is only concerned with spores and not the fungi themselves.

The publication entitled “The filamentous fungus Penicillium chrysogenum analysed via flow cytometry—a fast and statistically sound insight into morphology and viability” by L. Veiter et al., published in the journal Applied Microbial and Cell Physiology in 2019 (103: 6725-6735) states that it is possible to extract P. chrysogenum from its culture medium with a saline solution containing 50 g/l of a CaCl₂ solution of concentration 2.65 g/l solution, and having a concentration of KCl of 0.2 g/l, of KH₂PO₄ of 0.2 g/l, of MgCl·6H₂O of 0.1 g/l, of NaCl of 8 g/l and 0.764 g/l of Na₂HPO₄+2H₂O. The cells are stained with propidium iodide, which makes it possible to differentiate cells in which the cell is impaired. Fluorescein diacetate is also used to determine the metabolic activity of cells. This method is used for non-complex fungal culture media. It is not applicable to soils, which are too complex and for which the fungi, in particular hyphae, are closely bound to the particles of the medium.

The publication “Bioaerosol characterization by flow cytometry with fluorochrome” by Chen Pei-Shih et al. published in Journal of Environmental Monitoring in 2005 concerns the simultaneous detection of fungi and bacteria contained in air and water. The fluorophore used is SYTO-13 which has a fluorescence in the green range. This fluorophore makes it possible to detect and differentiate the fungi and bacteria contained in water and air. The bacteria detected are as follows: E. coli and endospores of B. subtilis; the fungi detected are as follows: C. famata and P. citrinum spores. This document says nothing about hyphae.

### Technical Problems Addressed

A first aim of the present invention is that of providing a flow cytometry method which makes it possible to simultaneously quantify the fungi and bacteria present in a solid in the divided state, in particular a soil sample, containing organic matter.

A further aim of the present invention is that of providing a method which furthermore makes it possible to simultaneously determine for the bacteria and for the fungi, the state of their cells, namely, latent, live or dead.

A further aim of the present invention is that of providing a method which makes it possible to account for the hyphae contained in the substrate.

### BRIEF DESCRIPTION OF THE INVENTION

The method relates to a method for the simultaneous detection of fungi and bacteria present in a divided solid containing cellulosic organic matter, whereby a solid-liquid extraction of said divided solid is carried out, a sample of said liquid obtained, optionally diluted, is introduced into a flow cytometer and a biparametric histogram is plotted giving for each point the diffracted light intensity and the reflected and refracted light intensity in such a way as to differentiate a first group of points attributable to fungi and a second group of points attributable to bacteria.

Characteristically, according to the invention, to said sample before introducing into said cytometer, at least a first fluorochrome capable of binding to DNA and emitting, following an excitation, in a wavelength equal to or greater than 599 and equal to or less than 657 nm is added, said sample is furthermore excited in such a way as to obtain the fluorescence of said first fluorochrome and said biparametric histogram is also plotted for the points corresponding to particles emitting in fluorescence in the emission wavelength of said first fluorochrome for the first group of points and for the second group of points and in that for each of said groups, a point cloud is determined which corresponds to a greater fluorescence intensity due to the first fluorochrome than that of the other points of said group and in that optionally the points of said cloud are counted, which correspond respectively to the cells of fungi or of bacteria.

The first fluorochrome being capable of binding to DNA, it makes it possible to distinguish the microorganisms from the background noise formed by the suspended particles.

Advantageously, said first fluorochrome is excited with a wave having a wavelength equal to or greater than 619 nm and equal to or less than 678 nm and in particular equal to 637 nm.

According to an embodiment that can be combined with each of the embodiments, said fluorescence due to said first fluorochrome is detected at a wavelength equal to 670 nm.

Advantageously, said first fluorochrome is capable of binding to the DNA of live cells and to the DNA of cells in which the wall is permeable, which correspond to dead cells.

Although both fungi and bacteria possess DNA, it was not obvious that a single fluorochrome would make it possible to separate them from the background noise.

Indeed, it is noteworthy that the cells of fungi are distinguished from those of bacteria. Fungi are eukaryotic whereas bacteria are prokaryotic. Furthermore, the wall of fungi is composed of chitin, whereas it is composed of peptidoglycans in bacteria. The permeability of the membrane to various fluorochromic markers is therefore not the same in bacteria and in fungi.

The solid may contain cellulose and cellulosic organic matter.

The solid may also contain mineral matter such as clays or sand, for example.

According to specific embodiment, the solid consists of cellulosic organic matter.

According to an embodiment that can be combined with each of the other embodiments, to said sample before introducing into said cytometer, at least a second and at least a third fluorochrome are also added, said second fluorochrome is capable of binding only to the DNA of cells in which the wall is permeable and said third fluorochrome becomes fluorescent by reacting with the esterases contained in live cells and, for at least one of said groups, a biparametric histogram is plotted giving the fluorescence intensity due to the second fluorochrome and the fluorescence intensity due to the third fluorochrome, three point clouds are determined on said histogram: a first point cloud for which the fluorescence of the third fluorochrome is the strongest, a second point cloud for which the fluorescence due to the second fluorochrome is the weakest and a third point cloud and it is inferred that the first point cloud corresponds to live bacteria/fungi, the second point cloud corresponds to fungi/bacteria in the latent state and the third point cloud corresponds to dead fungi/bacteria, optionally by counting the points in each cloud, the number of bacteria or fungi and their respective state are determined.

As explained above, it is surprising that a mixture of three fluorochromes succeeds in distinguishing the life states (latent, dead, live) of fungi and bacteria. Indeed, fungi and bacteria have different metabolisms which often do not allow them to be analyzed together. Furthermore, it is noteworthy that the method also accounts for hyphae, which are counted as an event (a point).

Advantageously, said first fluorochrome is selected from fluorochromes capable of binding to the DNA of cells and having a maximum fluorescence absorption wavelength equal to or greater than 599 nm and equal to or less than 657 nm and a maximum fluorescence emission wavelength equal to or greater than 619 nm and equal to or less than 678 nm and a quantum yield equal to or greater than 0.16 and equal to or less than 0.39.

In particular, said first fluorochrome may be selected from fluorochromes capable of binding to the DNA of cells and having a maximum fluorescence absorption wavelength of 652 nm and a maximum fluorescence emission wavelength of 676 nm and a fluorescence quantum yield on DNA of 0.27 and fluorochromes capable of binding to DNA and having a maximum fluorescence absorption wavelength of 657 nm and a maximum fluorescence emission wavelength of 673 nm and a fluorescence quantum yield on DNA of 0.17.

Such fluorochromes are marketed under the names SYTO62® and SYTO 63® by Thermofisher.

Advantageously, said second fluorochrome is selected from fluorochromes having a maximum fluorescence absorption wavelength of 547 nm and a maximum fluorescence emission wavelength of 570 nm and a fluorescence quantum yield on DNA of 0.9 and in that said third fluorochrome is selected from 5-carboxyfluorescein diacetate, 6-carboxyfluorescein diacetate, mixtures of 5-carboxyfluorescein diacetate and 6-carboxyfluorescein diacetate and 5,6 carboxylate fluorescein diacetate succinimidyl ester of the following general formula (1):

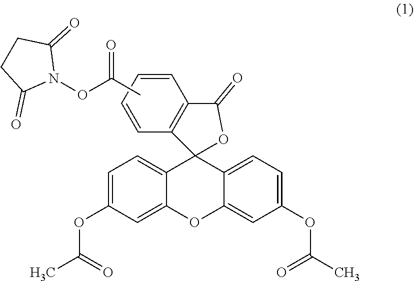

Advantageously, the second fluorochrome is the fluorochrome marketed by Thermofisher under the name SYTOX-orange.

Advantageously, the third fluorochrome is the cFDA mixture.

Advantageously, the first fluorochrome is SYTO62®, the second fluorochrome is SYTOX-orange and the third fluorochrome is the cFDA mixture

The divided solid containing cellulosic matter may be selected from top soil, potting soil, compost, manure, mulch, humus and mixtures therefore, in particular pairwise mixtures thereof.

The fungus or fungi and bacterium or bacteria contained in the divided solid are not limited according to the invention. They may consist of fungi and bacteria capable of breaking down cellulose but also other bacterial or fungi from animals. Indeed, when the solid is or contains manure, it is likely to also contain fecal bacteria.

Thus, the method according to the invention makes it possible to detect and quantify at least one fungus and preferably a mixture of fungi selected from microscopic fungi capable of forming mycorrhizae, fungi imperfecti, Lichtheimia corymbifera, mucor corymbifera, mucor mucedo, yeasts, and hyphae of macroscopic fungi and at least one bacterium or a mixture of bacteria selected from cytophaga spp, streptomyces spp, Bacillus radicola, Candida albicans, Phycomycetes Rhizopus, Penicillium spp, Aspergillus spp, verticillium, Helminthosporium, Fusarium, Cladosporium, actynomycetes, Nitrosomonas, Nitrosococcus, Azotobacter, Clostridium spp, Rhizobium, acidobacteria, Bacteroidetes, Firmicutes, Actinobacteria, Alphaproteobacteria and Betaproteobacteria, Saccharomyces spp, Pseudomonas spp, Staphylococcus aureus. E. coli, Micrococcus luteus, Bacillus megaterium, Bacillus polymyxa and Enterococcus faecium.

It is noteworthy that the method also makes it possible to detect and quantify hyphae which are groups of cells not separated by walls. As the fluorescence detected can be correlated with the quantity of DNA, the method also makes it possible to quantify hyphae, which are counted as several cells according to the number of nuclei contained therein.

Advantageously, the solid-liquid extraction is carried out with an aqueous sodium chloride solution containing 7 to 12 g/L of sodium chloride and in particular 8.5 g/L of sodium chloride and filtered with a cutoff threshold of 0.22 μm.

It is also surprising that despite the lack of surfactant, it is possible according to the invention to extract fungi and bacteria which are both closely bound to the particles of the divided solid. Furthermore, this method also surprisingly makes it possible to extract hyphae, which are closely bound physico-chemically to the particles of the solid.

Advantageously, said divided solid is screened before extraction in such a way as to only retain fragments less than or equal to 2 mm in size. It is likely, although the Applicant is not bound to this explanation, that screening makes it possible to already prepare the extraction of the fungi and bacteria and separate them already slightly from the particles of the divided solid.

Advantageously, said sample is diluted a first time to 1:10 or to 1:100 and then diluted to 5:100. Such a dilution facilitates detection and quantification.

The present invention also relates to a fluorochromic mixture containing a first fluorochrome selected from fluorochromes capable of binding to the DNA of cells and having a maximum fluorescence absorption wavelength equal to or greater than 599 nm and equal to or less than 657 nm and a maximum fluorescence emission wavelength equal to or greater than 619 nm and equal to or greater than 678 nm and a quantum yield equal to or greater than 0.16 and equal to or greater than 0.39 and mixtures thereof and in particular from fluorochromes capable of binding to the DNA of cells and having a maximum fluorescence absorption wavelength of 652 nm and a maximum fluorescence emission wavelength of 676 nm and a fluorescence quantum yield on DNA of 0.27 and fluorochromes capable of binding to DNA and having a maximum fluorescence absorption wavelength of 657 nm and a maximum fluorescence emission wavelength of 673 nm and a fluorescence quantum yield on DNA of 0.17 and mixtures thereof, a second fluorochrome selected from fluorochromes having a maximum fluorescence absorption wavelength of 547 nm and a maximum fluorescence emission wavelength of 570 nm and a fluorescence quantum yield on DNA of 0.9 and a third fluorochrome selected from 5-carboxyfluorescein diacetate, 6-carboxyfluorescein diacetate, mixtures of 5-carboxyfluorescein diacetate and 6-carboxyfluorescein diacetate and 5,6 carboxylate fluorescein diacetate succinimidyl ester of the following general formula (1):

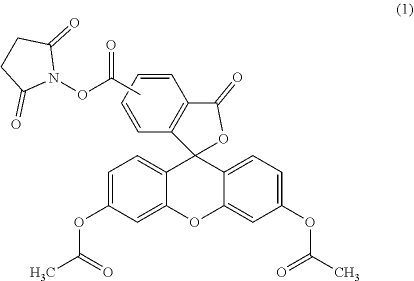

Advantageously, the mixture contains a greater concentration of third fluorochrome than the concentration of second and first fluorochrome and in that it contains a greater concentration of first fluorochrome than that of said second fluorochrome.

Indeed, the Inventors demonstrated that such a mixture could, with a single analysis with a flow cytometer, allow the detection, quantification and determination of the life state (latent, dead, live) of fungi and bacteria.

### Definitions

For the purposes of the invention, the term “divided state” denotes a powder, granules, fibers, particles and mixtures thereof.

For the purposes of the invention, the term “cellulosic organic matter” denote cellulose, hemicellulose, organic matter from the biological degradation of cellulose and/or hemicellulose and mixtures of at least two of these compounds. In particular, the term denotes a mixture of cellulose, hemicellulose and matter from the biological degradation of cellulose and hemicellulose.

The term “top soil” denotes any soil from humus-bearing surface horizons or deep horizons capable of being mixed with organic matter of plant origin, organic amendments and/or mineral matter. For the purposes of the invention, the term encompasses sandy top soil, silty top soil, calcareous top soil, humus-bearing top soil and loamy top soil which is a mixture of these four soils.

The term “compost” denotes the substrate obtained by decomposition on account of the organisms living in the soil or on the soil of plant and/or animal organic matter.

The term “potting soil” denotes a mixture of top soil and/or compost and/or manure.

The term “humus” denotes the upper layer of the soil present under trees, in particular in forests.

The term “mulch” denotes a divided material comprising or consisting of wood chippings and/or bark and/or needles and/or dried lawn cuttings and/or dead leaves and/or hay and/or straw and/or sawdust and/or hemp flakes and/or flax flakes and/or ramial chipped wood.

The term “simultaneous” means that the results can be obtained with a single pass of a single sample in a flow cytometer.

The term “fungi” encompasses all microscopic fungi and in particular microscopic fungi capable of breaking down cellulose and/or hemicellulose, hyphae of microscopic fungi and fungi imperfecti.

The term “bacteria” denotes according to the invention any bacteria capable of breaking down cellulose and/or hemicellulose and bacteria originating in animal tracts (plant-eating or meat-eating).

The acronym FSC refers to the signal corresponding to diffracted light (FSC: Forward Scatter); this signal depends on the size and surface area of the analyzed particle;

The acronym SSC (SSC: Side Scatter) refers to the signal corresponding to reflected and refracted light; this signal depends on the granularity and cellular complexity of the analyzed particle.

The acronym SSC-H refers to the signal strength corresponding to reflected and refracted light;

The acronym SSC-A refers to the intensity per unit of surface area of the signal corresponding to reflected and refracted light;

The acronyms FSC-H and FSC-A refer, respectively, to the intensity and intensity per unit of surface area of the signal corresponding to diffracted light;

The acronym cFDA or c-FDA denotes a mixture of 5-carboxyfluorescein diacetate and 6-carboxyfluorescein diacetate.

Throughout the application, the maximum fluorescence values in absorption and emission defining the fluorochromes are determined in the presence of DNA with a ratio of about 100 base pairs and in particular 100 base pairs of nucleic acid for a fluorochrome molecule in a Tris medium of pH 7.5 and an EDTA concentration equal to 1 mM. Throughout the application, the fluorescence quantum yield defining the fluorochromes is measured in the presence of DNA and expressed relative to the yield determined for cresyl violet in methanol.

Other features and advantages of the invention will become apparent upon reading the following description, with reference to the appended figures, which illustrate:

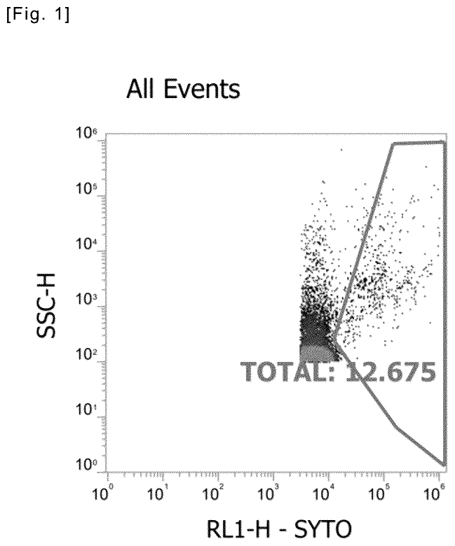

FIG. 1 is a biparametric histogram indicating for each point the fluorescence intensity emitted by the SYTO63® fluorochrome and the reflected and refracted light intensity;

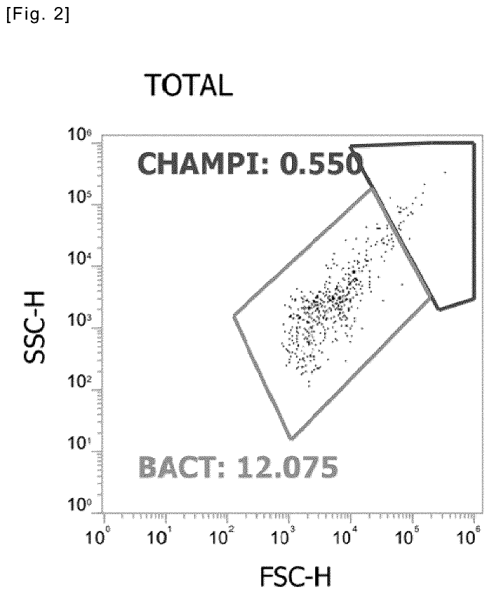

FIG. 2 is a biparametric histogram indicating for each point in the TOTAL window seen in FIG. 1 the diffracted light intensity and the reflected and refracted light intensity;

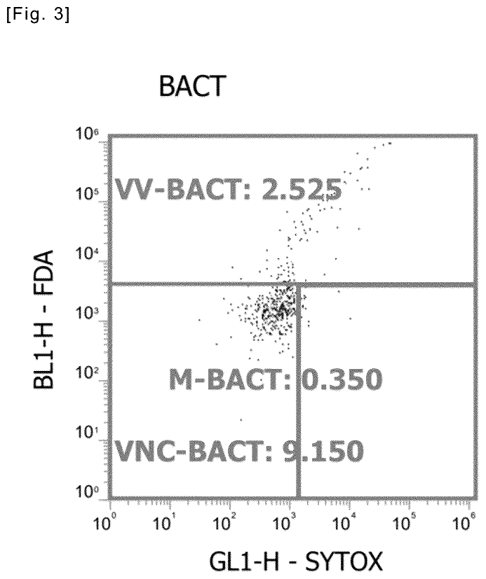

FIG. 3 is a biparametric histogram indicating for each point of the “bacteria” window seen in FIG. 2, the fluorescence intensity due to the SYTOX-orange fluorochrome and the fluorescence intensity due to the cFDA mixture; and

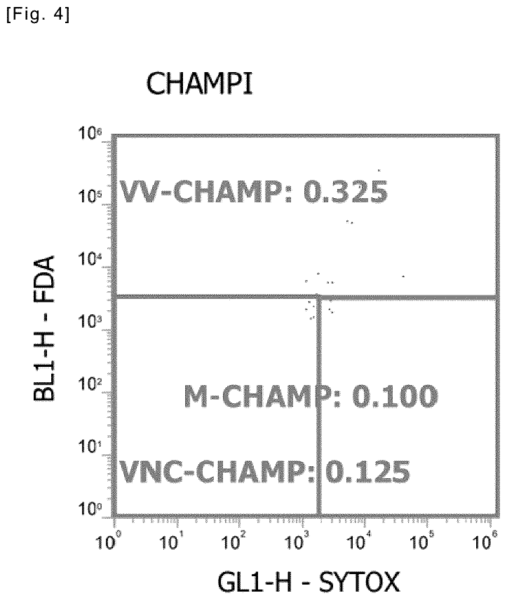

FIG. 4 is a biparametric histogram indicating for each point of the “fungus (champi)” window seen in FIG. 2, the fluorescence intensity due to the SYTOX-orange fluorochrome and the fluorescence intensity due to the cFDA mixture; and

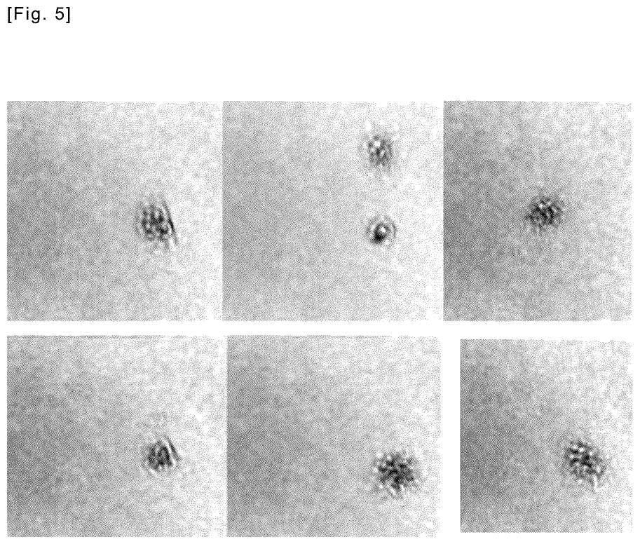

FIG. 5 represents photographs of fungal hyphae detected by the flow cytometer and indicated as an event (a point) on the results histogram; their state (latent, live, dead) may be determined with the method according to the invention.

### EXAMPLES

In the example below, the term “CHAMPI” is synonymous with fungi.

### Fluorochrome Mixture

Physiological saline solution (osmosed/ultra-pure water+NaCl at 8.5 g/L) is prepared then autoclaved and filtered before use (0.22 μm filter). SYTOX®-Orange (Thermofisher, 5 μM) and 5,6 carboxyfluorescein diacetate (c-FDA) are diluted in DMSO (respectively 12.5 μM and 2 g/L in final concentrations) then stored in a freezer; SYTO 63® (Thermofisher, 5 mM) is used directly in the labeling solution. The final fluorochrome concentrations in the labeling mixture are 7.5 μM of SYTO 63®, 0.05 μM of SYTOX®-Orange (SYTOX-or) and 10 mg/L of c-FDA (21.7 μM) in the sodium chloride solution.

### Sample Preparation

A sample of 2.5+/−0.1 g of soil (top soil) or fresh compost is screened at 2 mm; 22.5 mL of filtered physiological saline solution is then added. The solid-liquid extraction is then carried out using a rotary stirrer for 15 min. The sample is then left to settle overnight at ambient temperature. The supernatant is diluted to 1:10 (soil) or to 1:100 (compost), the dilutions are vortexed, a second dilution to 5:100 is finally produced in the labeling mix, the whole is vortexed. The labeling incubates for about 30 min protected from light before analysis.

### Materials Used

The cytometer used is an ATTUNE® NXT acoustic focusing cytometer (thermofisher scientific). This flow cytometer is equipped with 3 lasers: blue (488 nm), green (532 nm) and red (637 nm). The cytometer may be equipped with an automatic sample changer for reading 96-well microplates. This equipment is equipped with an acoustic flow focusing system making it possible to use a flow rate of up to 1000 μL/min.

The flow rate is set to 25 μL/min. The latter is slowed down when the bioburden is high. The data are collected on the following channels: FSC, SSC, BL1 (525/50) for c-FDA, GL1 (575/36) for SYTOX-orange and RL1 (670/14) for SYTO 62 or 63. The voltage values for each of these channels are 320V, 340V, 460V, 480V and 570V respectively. Different values may be applied.

There is no compensation problem to correct in this configuration.

### Processing of Results

In order to separate the microorganisms from the background noise, SYTO63® is used, thus SYTO63®-positive events are considered as microorganisms. SYTO62® may also be used.

Triple sample labeling and a “gating” (window determination) strategy made it possible to separate bacteria and fungi in soils and compost. In a first phase, most of the microorganisms are separated from the background noise by the SSC-H/RL1-H plot (670/14 nm) (see FIG. 1). The points for which fluorescence is obtained correspond to microorganisms because the SYTO63® fluorochrome binds to DNA and not to the suspended particles in the sample.

Then, thanks to the SSC-H/FSC-H plot applied to the TOTAL window seen in FIG. 1 and encompassing bacteria and fungi (microorganism containing DNA), bacteria and fungi are separated on account of their morphological and structural differences. Therefore, two windows are defined seen in FIG. 2: one CHAMPI window which contains the points attributable to fungi and one BACT window which contains the points attributable to bacteria. It was possible to validate these windows by comparing the results obtained with cytometry, microscopy and on Petri dishes. The analyses performed with microscopy and on Petri dishes enable a determination of the windows serving also for calibration for the same type of substrate, for example.

The different states of the microorganisms studied (total population, Bacteria and Fungi) are obtained by cross-referencing the data obtained by the different fluorochromes on each of the windows determined. Thus, for each window corresponding either to fungi, or to bacteria, biparametric histograms are produced giving the fluorescence intensities emitted for each of the two fluorochromes which are SYTOX-orange and the cFDA mixture. The “dead” state corresponds to a fluorescence emission due to SYTOX-Orange and an absence of fluorescence due to the cFDA mixture (SYTOX-or +/cFDA −). The “live latent (VNC viable non-culturable)” state corresponds to an absence of or low fluorescence emission due to SYTOX-orange and an absence of or low fluorescence emission due to the cFDA mixture (SYTOX-Orange −/c-FDA −). The “live active/vital” state corresponds to the points exhibiting an absence of fluorescence emission due to SYTOX orange and a fluorescence emission due to the cFDA mixture (SYTOX-Orange −/c-FDA+).

The method according to the invention thus makes it possible to carry out at the same time, with the same sample and with a single pass in the cytometer: separation of the microorganisms of interest (fungi and bacteria) from the background noise, separation of live microorganisms from dead microorganisms, within the live microorganism population, separating physiologically active microorganisms from dormant microorganisms (latent state);

It also simultaneously provides all the vitality and viability information. The method is rapid, with active labeling in about 15 minutes, inexpensive and can be industrialized for high-throughput analysis. Furthermore, as seen in the photographs of FIG. 5, the method makes it possible to extract hyphae, detect them by flow cytometry by labelling and determine their life state (latent, dead, active) via triple labeling.

Of course, the invention is described above by way of example. It is understood that a person skilled in the art is capable of creating various alternative embodiments of the invention without for all that leaving the scope of the invention.

1. A method for the simultaneous detection of fungi and bacteria present in a divided solid containing cellulosic organic matter, whereby a solid-liquid extraction of the divided solid is carried out, a sample of the liquid obtained, optionally diluted, is introduced into a flow cytometer and a biparametric histogram is plotted giving for each point the diffracted light intensity and the reflected and refracted light intensity in such a way as to differentiate a first group of points attributable to fungi and a second group of points attributable to bacteria, characterized in that, to the sample before introducing into the cytometer, at least a first fluorochrome capable of binding to DNA and emitting, following an excitation, in a wavelength equal to or greater than 599 and equal to or less than 657 nm is added, in that the sample is furthermore excited in such a way as to obtain the fluorescence of the first fluorochrome, in that the biparametric histogram is also plotted for the points corresponding to particles emitting in fluorescence in the emission wavelength of the first fluorochrome for the first group of points and for the second group of points and in that for each of the groups, a point cloud is determined which corresponds to a greater fluorescence intensity due to the first fluorochrome than that of the other points of the group and in that optionally the points of the cloud are counted, which correspond respectively to the cells of fungi or of bacteria.

2. The method of claim 1, characterized in that the first fluorochrome is excited with a wave having a wavelength equal to or greater than 619 nm and equal to or less than 678 nm and in particular equal to 637 nm.

3. The method of claim 1, characterized in that the fluorescence due to the first fluorochrome is detected at a wavelength equal to 670 nm.

4. The method of claim 1, characterized in that the first fluorochrome is selected from fluorochromes capable of binding to the DNA of cells and having a maximum fluorescence absorption wavelength equal to or greater than 599 nm and equal to or less than 657 nm and a maximum fluorescence emission wavelength equal to or greater than 619 nm and equal to or less than 678 nm and a quantum yield equal to or greater than 0.16 and equal to or less than 0.39 and in particular from fluorochromes capable of binding to the DNA of cells and having a maximum fluorescence absorption wavelength of 652 nm and a maximum fluorescence emission wavelength of 676 nm and a fluorescence quantum yield on DNA of 0.27 and fluorochromes capable of binding to DNA and having a maximum fluorescence absorption wavelength of 657 nm and a maximum fluorescence emission wavelength of 673 nm and a fluorescence quantum yield on DNA of 0.17.

5. The method of claim 1, characterized in that, to the sample before introducing into the cytometer, at least a second and at least a third fluorochrome are also added, in that the second fluorochrome is capable of binding only to the DNA of cells in which the wall is permeable and a third fluorochrome which becomes fluorescent by reacting with the esterases contained in live cells and, in that, for at least one of the groups, a biparametric histogram is plotted giving the fluorescence intensity due to the second fluorochrome and the fluorescence intensity due to the third fluorochrome, two or three point clouds are determined on the histogram: a first point cloud for which the fluorescence of the third fluorochrome is the strongest, a second point cloud for which the fluorescence due to the second fluorochrome is the weakest and a third point cloud and it is inferred that the first point cloud corresponds to live bacteria/fungi, the second point cloud corresponds to fungi/bacteria in the latent state and the third point cloud corresponds to dead fungi/bacteria, by counting the points in each cloud, the number of bacteria or fungi and their respective state are determined.

6. The method of claim 1, characterized in that the second fluorochrome is selected from fluorochromes having a maximum fluorescence absorption wavelength of 547 nm and a maximum fluorescence emission wavelength of 570 nm and a fluorescence quantum yield on DNA of 0.9 and in that the third fluorochrome is selected from 5-carboxyfluorescein diacetate, 6-carboxyfluorescein diacetate, mixtures of 5-carboxyfluorescein diacetate and 6-carboxyfluorescein diacetate and 5,6 carboxylate fluorescein diacetate succinimidyl ester of the following general formula (1):

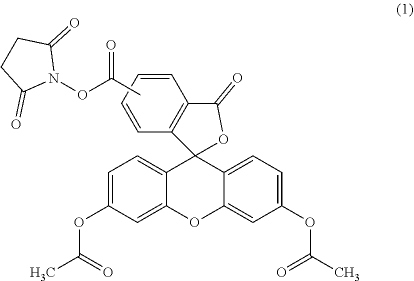

7. The method of claim 1, characterized in that the divided solid containing cellulosic matter is selected from top soil, potting soil, compost, manure, mulch, humus and mixtures therefore, in particular pairwise mixtures thereof.

8. The method of claim 1, characterized in that it makes it possible to detect and quantify at least one fungus and preferably a mixture of fungi selected from microscopic fungi capable of forming mycorrhizae, fungi imperfecti, Lichtheimia corymbifera, mucor corymbifera, mucor mucedo, yeasts, and hyphae of macroscopic fungi and at least one bacterium or a mixture of bacteria selected from cytophaga spp, streptomyces spp, Bacillus radicola, Candida albicans, Phycomycetes Rhizopus, Penicillium spp, Aspergillus spp, verticillium, Helminthosporium, Fusarium, Cladosporium, actynomycetes, Nitrosomonas, Nitrosococcus, Azotobacter, Clostridium spp, Rhizobium, acidobacteria, Bacteroidetes, Firmicutes, Actinobacteria, Alphaproteobacteria and Betaproteobacteria, Saccharomyces spp, Pseudomonas spp, Staphylococcus aureus. E. coli, Micrococcus luteus, Bacillus megaterium, Bacillus polymyxa and Enterococcus faecium.

9. The method of claim 1, characterized in that the solid-liquid extraction is carried out with an aqueous sodium chloride solution containing 7 to 12 g/L of sodium chloride and in particular 8.5 g/L of sodium chloride and filtered with a cutoff threshold of 0.22 μm.

10. The method of claim 1, characterized in that the divided solid is screened before extraction in such a way as to only retain fragments less than or equal to 2 mm in size.

11. A fluorochromic mixture containing a first fluorochrome selected from fluorochromes capable of binding to the DNA of cells and having a maximum fluorescence absorption wavelength equal to or greater than 599 nm and equal to or less than 657 nm, a maximum fluorescence emission wavelength equal to or greater than 619 nm and equal to or greater than 678 nm and a quantum yield equal to or greater than 0.16 and equal to or greater than 0.39 and mixtures thereof and in particular from fluorochromes capable of binding to the DNA of cells and having a maximum fluorescence absorption wavelength of 652 nm and a maximum fluorescence emission wavelength of 676 nm and a fluorescence quantum yield on DNA of 0.27 and fluorochromes capable of binding to DNA and having a maximum fluorescence absorption wavelength of 657 nm and a maximum fluorescence emission wavelength of 673 nm and a fluorescence quantum yield on DNA of 0.17 and mixtures thereof, a second fluorochrome selected from fluorochromes having a maximum fluorescence absorption wavelength of 547 nm and a maximum fluorescence emission wavelength of 570 nm and a fluorescence quantum yield on DNA of 0.9 and a third fluorochrome selected from 5-carboxyfluorescein diacetate, 6-carboxyfluorescein diacetate, mixtures of 5-carboxyfluorescein diacetate and 6-carboxyfluorescein diacetate and 5,6 carboxylate fluorescein diacetate succinimidyl ester of the following general formula (1):

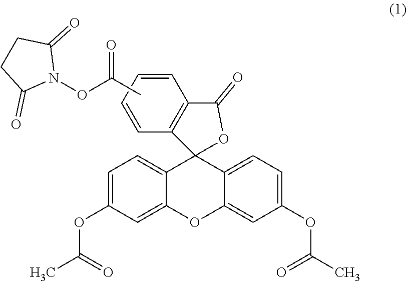

12. The fluorochromic mixture of claim 11, characterized in that it contains a greater concentration of third fluorochrome than the concentration of second and first fluorochrome and in that it contains a greater concentration of first fluorochrome than that of the second fluorochrome.
